(19) United States
(12) Patent Application Publication
(10) Pub. No.: US 2013/0338217 A1
(43) Pub. Date: Dec. 19, 2013

(54) ANTIBIOTIC COMPOSITION INCLUDING PHLOROTANNIN COMPOUND DERIVED FROM EISENIA BICYCLIS AS EFFECTIVE COMPONENT

(71) Applicant: PUKYONG NATIONAL UNIVERSITY INDUSTRY-UNIVERSITY C, Busan (KR)

(72) Inventors: Se-Kwon KIM, Busan (KR); Young-Mog Kim, Busan (KR); Myung-Suk Lee, Busan (KR); Sung-Hwan Eom, Busan (KR)

(73) Assignee: Pukyong National University Industry-University Cooperation Foundation, Busan (KR)

(21) Appl. No.: 13/664,000

(22) Filed: Oct. 30, 2012

(30) Foreign Application Priority Data
Jun. 18, 2012 (KR) 10-2012-0064947

(51) Int. Cl. A61K 31/357; A61K 45/06

(52) U.S. Cl. CPC A61K 31/357; A61K 45/06
USPC 514/452; 549/359; 549/358

(57) ABSTRACT

There is provided an antibiotic composition including Eisenia bicyclis-derived phlorotannin compound, in which the antibiotic composition includes a phlorotannin compound selected from 7-phloroeckol, fucofuroeckol-A, and dioxinodehydroeckol, or mixture thereof, which are derived from Eisenia bicyclis, as an effective component. The compounds described above exhibit an antibiotic activity against an antibiotic-resistant bacterium. In addition, the compound described above can exhibit a synergetic effect by using in combination with β-lactam-based antibiotic composition by recovering an antibiotic activity of β-lactam-based antibiotic, such as ampicillin, penicillin, and oxacillin against MRSA.

### CROSS-REFERENCE TO RELATED APPLICATIONS

This application claims the priority of Korean Patent Application No. 2012-64947 filed on Jun. 18, 2012, in the Korean Intellectual Property Office, the disclosure of which is incorporated herein by reference.

### BACKGROUND OF THE INVENTION

1. Field of the Invention

The present invention relates to an antibiotic (antimicrobial) composition including a phlorotannin compound derived from Eisenia bicyclis as an effective component, and more particularly, to an antibiotic composition including a phlorotannin compound as an effective component, in which the antibiotic composition has excellent antibiotic activity against methicillin-resistant Staphylococcus aureus (MRSA).

2. Description of the Related Art

In general, antibiotic is a generic term for antimicrobial agents, and especially, means a substance having an antibacterial function against bacteria, specifically, a substance having an excellent antibacterial function by inhibiting a system for synthesizing cell wall, protein, or the like by bacteria, or a material prepared from this substance. The components of antibiotic are mainly extracts derived from mold, and today, are frequently used for treating diseases caused by bacterial infections, or the like. The most representative antibiotic is penicillin prepared by Alexander Fleming, the British doctor, in 1928. Penicillin is the first antibiotic prepared for responding to bacteria in earnest by mankind. The representative antibiotic developed after the penicillin development is methicillin that is accepted as antibiotic having excellent effect as compared with penicillin. Methicillin is prepared by partially changing a chemical structure of penicillin.

The methicillin is classed as the β-lactam antibiotics, which work by binding with penicillin-binding proteins (PBPs) and then removing activities of the proteins.

An antibiotic-resistant bacterium is a bacterium that is resistant to a specific antibiotic, so that the specific antibiotic has no effect on the bacterium. For example, there is penicillin-resistant Staphylococcus aureus, in which the penicillin has no effect. Above this, there is also methicillin-resistance Staphylococcus aureus (MRSA) that has been initially reported in the academic world in 1961, and since then, is main hospital infection bacterial all over the world. It has been revealed that the MRSA has a special gene that may be resistant to antibiotic, such as penicillin and methicillin. It is reported that the MRSA does not infect to healthy people, and mainly infects to patients with compromised immune systems or patients after the operation. It is also reported that if people are infected by the MRSA, they die of septicaemia or pneumonia.

These days, it is on the increase that the MRSA infects to healthy people. The medical world or public health world of the United States is concerned about the increase of the infection by a pathogenic bacterium that is not treated by various antibiotics, such as MRSA (multiple drug resistance pathogenic bacteria).

### SUMMARY OF THE INVENTION

An aspect of the present invention provides an antibiotic composition having excellent antibiotic activity against various bacteria, particularly strong antibiotic activity against methicillin-resistant Staphylococcus aureus (MRSA), in which the antibiotic composition includes a phlorotannin compound derived from Eisenia bicyclis as an effective component.

In order to achieve the object, the present invention provides an antibiotic composition including a phlorotannin compound derived from Eisenia bicyclis as an effective component. Especially, the antibiotic composition may be used as a composition for preventing or treating a disease caused by an infection of methicillin-resistant Staphylococcus aureus, or a composition for an animal feed.

According to an aspect of the present invention, an antibiotic composition of the present invention has a phlorotannin compound derived from Eisenia bicyclis as an effective component, in which the phlorotannin compound is selected from the group consisting of 7-phloroeckol, fucofuroeckol-A, dioxinodehydroeckol, and a mixture thereof.

According to another aspect of the present invention, the phlorotannin compound may be obtained from extract, fractions, microbial fermentation materials, or purified materials of Eisenia bicyclis.

According to still another aspect of the present invention, the phlorotannin compound may be obtained from n-hexane fractions, dichloromethane fractions, ethyl acetate fractions, and n-butanol fractions of methanol crude extract of Eisenia bicyclis, and may be preferably obtained from the ethyl acetate fractions.

According to still another aspect of the present invention, the phlorotannin compound has antibiotic activity against methicillin-resistant Staphylococcus aureus (MRSA).

According to still another aspect of the present invention, the phlorotannin compound inhibits a synthesis of PBP2a protein in methicillin-resistant Staphylococcus aureus (MRSA).

According to still another aspect of the present invention, the antibiotic composition further includes β-lactam-based antibiotics, such as ampicillin, penicillin, and oxacillin.

According to still another aspect of the present invention, the antibiotic composition may be used as pharmaceutically, cosmetically, and foodially composition, but the present invention is not limited thereto.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a stage drawing of a methanol-soluble extract and sequential fraction of Eisenia bicyclis;

FIG. 2 is a flowchart of compounds 1-6 from the ethyl acetate-soluble extract of Eisenia bicyclis;

FIG. 3 is structures of the phlorotannin compounds from Eisenia bicyclis;

FIGS. 4a and 4b are ¹H-NMR spectrum (a) and ¹³C-NMR spectrum (b) of compound 1 in DMSO-d₆, respectively;

FIGS. 5a and 5b are ¹H-NMR spectrum (a) and ¹³C-NMR spectrum (b) of compound 2 in DMSO-d₆, respectively;

FIGS. 6a and 6b are ¹H-NMR spectrum (a) and ¹³C-NMR spectrum (b) of compound 3 in DMSO-d₆, respectively;

FIGS. 7a and 7b are ¹H-NMR spectrum (a) and ¹³C-NMR spectrum (b) of compound 4 in DMSO-d₆, respectively;

FIGS. 8a and 8b are ¹H-NMR spectrum (a) and ¹³C-NMR spectrum (b) of compound 5 in DMSO-d₆, respectively;

FIGS. 9a and 9b are ¹H-NMR spectrum (a) and ¹³C-NMR spectrum (b) of compound 6 in DMSO-d₆, respectively;

FIGS. 10A and 10B are Scanning electron microscopic profiles on the antimicrobial effect of phlorofucofuroeckol-A against methicillin-resistant Staphylococcus aureus (MRSA) for (A) KCCM 40510 and (B) KCCM 40511;

FIG. 11a is a photograph illustrating an effect of phlorofucofuroeckol-A on the mRNA expression of mecA, mecI, mecR1, femA and GAPDH genes; and FIG. 11b shows graphs illustrating an effect of phlorofucofuroeckol-A on the mRNA expression of i) mecA, ii) mecI, iii) iv) mecR1, and v) femA genes; and

FIG. 12 is a photograph (A) and graph (B) illustrating an effect of phlorofucofuroeckol-A on the expression of PBP2a protein against MRSA strains.

### DETAILED DESCRIPTION OF THE PREFERRED EMBODIMENT

Exemplary embodiments of the present invention will now be described in detail with reference to the accompanying drawings.

All the technical terms used for the present invention have the following definitions, as long as the terms are differently defined, and have the same meanings as terms that are generally understood by those skilled in the art. In addition, a preferable method and sample are disclosed in the present specification, but things that are slimier or equal to the same are included in the range of the present invention. The contents of all the publications disclosed in the present specification as a reference are introduced in the present invention.

The term, an “antibiotic composition” may means an antibiotic that is a general term of an antimicrobial agent, and for example, means an antifungal agent, a germicide, preservative, a preserved agent, or a fungistat. Preferably, it means a material capable of inhibiting or suppressing growth and a vital function of Gram-positive bacterial and Gram-negative bacterial.

The term, “extract” includes conceptions including all of the extract solutions, factions, and purified materials obtained from all the stages in extracting, fractioning, or purifying, or diluent solutions, concentration solutions, and dried materials thereof.

The term, “approximately” means an amount, a level, a value, a number, a frequency, a percentage, dimension, size, weight, and length that are changed to the degree of 30, 25, 20, 25, 10, 9, 8, 7, 6, 5, 4, 3, 2, or 1% with respect to a reference amount, level, value, number, frequency, percentage, dimension, size, weight, and length.

Unless it is not differently needed in the context through the present specification, the expressions such as “include” and “including” include suggested steps or components, or the groups of steps or components, but it should be understood that they do not exclude other arbitrary steps or components, or the groups of other arbitrary steps or components.

Hereinafter, the present invention will be described in detail.

The present invention relates to an antibiotic composition including a compound isolated from Eisenia bicyclis, and more particularly, the phlorotannin compound isolated from Eisenia bicyclis, brown algae, has an antibiotic activity against antibiotic-resistant bacteria by involving in inactivating penicillin binding protein 2a (PBP2a) of MRSA or inhibiting a synthesis of the same protein.

Eisenia bicyclis that is a raw material for the present invention is an oceanophyte belonging to brown algae, and also seaweed belonging to sea mustard of sea cabbage. Generally, its length is 1 to 1.5 m or longer as a larger one, but depends on the depth of water in its habitat. It is perennial by four years, and its oil body is mainly shown up in spring. Generally, it can be collected in Japan and Korea, particularly, Ulleungdo and Dokdo's eastern coast. The collected Eisenia bicyclis is extracted with solvent, and desired extract can be purified by using a purification method that is known by those skilled in the art. There are many researches on various physiological activity such as conventional antioxidant activity, anti-inflammatory activity, and anticancer activity. However, the research on the antibiosis is yet insignificant.

Accordingly, the present invention confirmed an antibiotic activity of Eisenia bicyclis against methicillin-resistant staphylococcus aureus (MRSA) that is one of the main cause bacterium of the pathogenic infectious disease and isolated specific phlorotannin compounds (7-phloroeckol, fucofuroeckol-A, dioxinodehydroeckol) as a single material having an anti-MRSA activity from Eisenia bicyclis.

The solvent suitable for the extract may include water or an organic solvent, and for example, water, or alcohols with 1 to 4 of carbon number such as methanol, ethanol, propanol, isopropanol, and butanol, or various solvents such as acetone, ether, benzene, chloroform, ethyl acetate, methylene chloride, dichloromethane, hexane, and cyclohexane, or mixed solvents thereof.

In addition, since then, the extracts extracted by using the solvents may be further subjected to a fraction process by using a solvent selected from the group consisting of hexane, butanol, dichloromethane, acetone, ethylacetate, ethyl ether, chloroform, water, and a mixture thereof. Preferably, the fraction solvent may be preferably dichloromethane, ethyl acetate, and n-butanol. The solvent upon the fraction may include at least one solvent. The Eisenia bicyclis extract according to the present invention may be prepared according to a general method of preparing a seaweed extract, and specifically, the general method may include a macerating extraction method, a digesting extraction method, a hot-water extraction method, or the like. A general extraction machine, ultrasonicator, or homogenizer may be used. In addition, since then, the solvents in the extract extracted by using the solvents described above may be removed through performing a filtration process, a concentration process, or drying process, and also performing all the filtration, concentration, and drying processes. Specifically, the filtration process may use a filter paper or a decompression filter, the concentration process may be performed by using a vacuum concentrator, for example, a rotary evaporator, and the drying process may be performed by using a freeze drying process, for example. Furthermore, the Eisenia bicyclis extract obtained may be stored in a deep freezer until it is used, water in the extract may be completely removed through a concentration and freeze-drying process. The Eisenia bicyclis extract without water may be used in a type of powder, or may be used through dissolving the powder in distilled water or a general solvent.

As an example of the present invention, the Eisenia bicyclis extract may be prepared by removing salt and impurities in Eisenia bicyclis, drying to prepare a dried sample, adding to the dried sample to prepare a crude extraction liquid, and then vacuum-concentrating the crude extraction liquid. At this time, the removing of salt and impurities in Eisenia bicyclis may be performed by washing it with running water and the dried sample may be prepared by drying the Eisenia bicyclis without the salt and impurities, and then grinding the dried Eisenia bicyclis. Furthermore, the dried Eisenia bicyclis sample may be stored in a deep freezer until it is used. In addition, water in the Eisenia bicyclis extract may be completely removed through concentrating and freeze-drying the obtained extraction liquid, and the Eisenia bicyclis extract without water may be used in a type of powder or by dissolving it in a general solvent.

The Eisenia bicyclis-derived phlorotannins (7-phloroeckol, fucofuroeckol-A, and dioxinodehydroeckol) according to the present invention exhibit an antimicrobial activity against MRSA. The present inventors investigated a synergic effect generated by using the phlorotannin compounds in combination with β-lactam-based antibiotics without sensitivity to MRSA in order to investigate an antimicrobial mechanism of the phlorotannin compounds against MRSA. As a result, it was confirmed that an antimicrobial activity of β-lactam-based antibiotics such as ampicillin, penicillin, and oxacillin against MRSA was recovered (see Experimental Examples 9 and 10). The result described above means that the phlorotannin compounds of the present invention involves in inactivating penicillin binding protein 2a (PBP2a) protein of MRSA relating to β-lactam-based antibiotic-resistant, or inhibiting the synthesis of the protein described above. In order to investigate such a deduction with a molecular biologically technique, effects of the phlorotannin compounds according to the present invention on a protein synthesis and a transcription of genes relating to β-lactam-based antibiotic-resistant of MRSA and relevant gene relating to the synthesis of PBP2a protein were investigated by using a RT-PCR and a western blot (see Experimental Example 11). It was observed that the phlorotannin compounds of the present invention inhibited the transcription of gene of mec operon (mecA, mecI, mecR1) involving in the synthesis of PBP2a depending on the concentration thereof. Furthermore, from the western blot, it was confirmed that the phlorotannin compounds inhibited the synthesis of PBP2a that is a final product generated by mecA gene depending on the concentration thereof. From such a result, it is found that the phlorotannin compounds according to the present invention can recover antibiotic sensitivity to β-lactam-based antibiotics by directly inhibiting the synthesis of PBP2a protein that suppresses a transport of the β-lactam-based antibiotics into a cell in MRSA.

The antibiotic composition according to the present invention may include 0.01 wt % to 99.99 wt o, and preferably 0.1 wt % to 99 wt % of the Eisenia bicyclis extract based on the total weight of the composition, and the content of the effect components may be controlled according to a use method and use purpose of the antibiotic composition.

The antibiotic composition may have an antimicrobial activity against pathogenic microorganism, especially, various pathogenic bacteria, and more particularly, may have an antimicrobial activity against methicillin-resistant Staphylococcus aureus (MRSA), E. coli, Klebsiella. sp, Bacillus. sp, Micrococcus. sp, Staphylococcus. sp, Enterobacter. sp, Vibrio. sp, or Edwardsiella. sp. Preferably, it may be the antibiotic composition having an antimicrobial activity against methicillin-resistant Staphylococcus aureus (MRSA), E. coli, Klebsiella pnermoniae, Bacillus subtilis, Micrococcus leuteus, Staphylococcus aureus, Enterobacter aerogenes, Vibrio parahemolyticus, or Edwardsiella tarda, and more preferably, having an antimicrobial activity against methicillin-resistant Staphylococcus aureus (MRSA).

Such methicillin-resistant Staphylococcus aureus (MRSA) is a pathogenic bacterium belonging to a gram-positive micrococcus, and exhibits resistance against methicillin, β-oxacillin, or nafcillin that is a pencillin-based antibiotic among them. Such methicillin-resistant Staphylococcus aureus (MRSA) exhibits resistance against most of β-lactam-based antibiotics including cephalosporins and imipenem, and often exhibits resistance against most the antibiotics, such as aminoglycoside antibiotics and quinolones. It is reported that such methicillin-resistant Staphylococcus aureus (MRSA) causes a severe infection such as pneumonia and sepsis as well as a skin infection such as an abscess or an infection of wound. It is reported that in order to treat the infections described above, only a very limited antibiotic such as vancomycin or teicoplanin can be used.

It is reported that the diseases infected by such methicillin-resistant Staphylococcus aureus (MRSA) include an infectious disease of a respiratory system, such as pneumonia, lung suppuration, and empyema thoracis, sepsis, bacteremia, infectious endocarditis, an infectious disease of intestine, an intractable bedsore, an infection of wound, or the like.

Such methicillin-resistant Staphylococcus aureus (MRSA) may cause an infection by settling in any part of human body, and may be possible to infect through an airborne transmission as well as a contact transmission. Especially, it is reported that it is well settled in a surgical patient or a burn patient with a damaged skin defense mechanism, so that a server infection may be caused, and also in the case of a medical personnel working in a hospital for a long period time, the MRSA has a high intranasal settlement rate, so that it can be possible to infect through the airborne transmission.

Such E. coli is a bacteria living in an intestine of human or an animal, especially, often in a large intestine. In the case of organs other than the intestine, it may cause a disease, especially, cystitis, pyelitis, sepsis, or the like. In the case of E. coli causing contagious diarrhea, it is referred to as a pathogenic E. coli.

Such Klebsiella pnermoniae is a pathogenic bacillus that causes pneumonia, and a gram-negative short bacillus.

Such Vibrio parahemolyticus is a main bacterium causing a food poisoning, and is often found in seawater, deposit soil, living organism of an invertebrate, and the like of coast and estuarine deposit. Often, it infects cockles, Manila clam, sea mustard, ark clam, shrimps, small octopus, a frigate mackerel, goby, or the like. Vibrio parahemolyticus attached the above-described seafood may contaminate other foods through a refrigerator, cooking utensils such as a cutting board, a dishtowel, and a knife, and hands of cooker. If human intakes the contaminated food described above, a food poisoning, and the like may be caused.

Such Edwardsiella. sp is a gram-negative bacterium, an intestinal bacteria causing disability of a digestive tract mainly in fish, and especially, Edwardsiella tarda causes an Edward's syndrome. Such an Edward's syndrome is caused in large fish of two years old or more in the period of high water temperature, and its appearance syndrome includes an ulcer of a head part, a distension or redness of abdominal region, white turbidity or projection of eyes, or the like and its inside syndrome is characterized in that ascites are filled, and gonad condion, especially, ovary turns red or hardens, or the like. An accumulated mortality rate of the disease described above is 20% to 30%.

The antibiotic composition according to the present invention may exhibit an antibiotic activity by directly applying to an animal including human, lower the mortality rate of fish, shellfish, or the like, increase their productivities, and also prevent a secondary infection to other animals, like human, ingesting marine organisms.

Such animals are a living group that corresponds to plants, and often ingests organic matters as a nutrient, of which the digestive organs, the excretion organs, and the respiratory organs are differentiated. A specific example of such animals may include an echinoderm, crustaceans, mollusks, fish, amphibian, reptile, tidal current, and mammals, and preferably, an echinoderm such as spatangoids or holothuroids, crustacean such as crabs, shrimps, and spiny lobster belonging to an arthropod, mollusks such as cephalopods, gastropods, or bivalves, fish such as a red sea-bream, snapper, cod, flat fish, and halibut, fowls including a poultry farm such as pheasant or chicken, a mammal such as human, pigs, cows, and a goat.

The antibiotic composition according to the present invention may include a sugar, protein, lipid, vitamins, and minerals in addition to the phlorotannin compound derived from Eisenia bicyclis.

The sugar may be properly selected according to its use purpose and use, and an example of the sugar may include, but is not particularly limited to, honey, dextrin, sucrose, palatinose, glucose, fructose, starch syrup, sugar alcohol, sorbitol, xylitol, and maltitol. The protein may be properly selected according to its use purpose and use, and an example of the protein may include, but is not particularly limited to, milk-derived proteins such as casein and whey protein, soybean protein, animal-derived enzyme such as pepsin and trypsin of these protein, and hydrolysate by neutrase and alkilase. The lipid may be properly selected according to its use purpose and use, and an example of the liquid may include, but is not particularly limited to, monovalent saturated fatty acids, all kinds of plants-derived oils such as sunflower oils including polyvalent unsaturated fatty acids, rapeseed oils, olive oils, safflower oils, corn oils, soybean oils, palm oils, and coconut oils, middle-chain fatty acid, EPA, DHA, soybean-derived phospholipid, and milk-derived phospholipid. The vitamins are not limited particularly, and may be properly selected according to its use purpose and use. An example of the vitamins may include, but is not particularly limited to, potassium phosphate, potassium carbonate, potassium chloride, sodium chloride, calcium lactate, calcium gluconate, calcium pantothenate, calcium casein, magnesium chloride, ferrous sulfate, and sodium hydrogen carbonate.

With respect to any one of the sugar, protein, lipid, vitamins, and minerals, they may further include other components, but not limited to the above specific examples as long as the antibiotic activity of the composition is maintained. The content of each of them is not limited as long as it is maintained, and preferably may by 0.1 wt % to 15 wt %, and preferably 0.5 wt % to 10 wt % relative to the total composition.

The antibiotic composition according to the present invention may be used for various purposes and uses, and specifically, it may be used for compositions for preventing or treating a disease caused by a pathogenic microbial infection, compositions for animal feeds, additives for animal feeds, foods, food additives, drinks, cosmetic compositions, cosmetic additives, or a cleansing agent.

Furthermore, the present invention provides a composition for preventing or treating composition a disease caused by a methicillin-resistant Staphylococcus aureus (MRSA) infection, the composition including the antibiotic composition described above.

The diseases infected by such methicillin-resistant Staphylococcus aureus (MRSA) means all kinds of infectious diseases that are possible to be caused by Staphylococcus aureus infection, and preferably may be an infectious disease of a respiratory system, such as pneumonia, lung suppuration, and empyema thoracis, sepsis, bacteremia, infectious endocarditis, enteritis, an infectious disease of intestine, an intractable bedsore, an infection of wound, or the like, and more preferably may be any one selected from the group consisting of pneumonia, sepsis, bacteremia, infectious endocarditis, and enteritis.

The composition for preventing or treating the diseases infected by the methicillin-resistant Staphylococcus aureus (MRSA) according to the present invention may further include a psychopharmacologically acceptable carrier or excipient according to a dosage form or use method in addition to the phlorotannin compound derived from Eisenia bicyclis. The composition according to the present invention may be administered along or along with other a pharmaceutical composition in order to improve an effect as a medicine for an infection caused by the methicillin-resistant Staphylococcus aureus. In this case, the content of Eisenia bicyclis extract may be 0.001 wt % to 99 wt % in the composition for preventing or treating the diseases infected by the methicillin-resistant Staphylococcus aureus. When the content of an effective component in the composition is less than 0.001 wt %, it may be required to administer the composition in large quantities in order for an effective effect, and on the other hand, when it is greater than 99 wt %, it may be uneconomical because the effect may be constant as compared with the use amount. In addition, the content of the Eisenia bicyclis extract in the composition for preventing or treating the diseases infected by the methicillin-resistant Staphylococcus aureus (MRSA) may be properly controlled according to the use method and use purpose.

The composition for preventing or treating the diseases infected by the methicillin-resistant Staphylococcus aureus (MRSA) may be administered orally or parenterally, and preferably orally administered. However, the dosage form may depend on the use method, and thus is not limited to the following description. An example of the dosage form may include plasters, granules, lotions, powders, syrups, liquids and solutions, aerosols, ointments, fluidextracts, emulsions, suspensions, infusions, tablets, injections, capsules, pills, and the like.

Further, the composition for preventing or treating the diseases infected by the methicillin-resistant Staphylococcus aureus may be desirably formulated by using the proper known method in the related art or the method disclosed in Remington's Pharmaceutical Science (The latest issue) (Mack Publishing Company, Easton Pa.).

With respect of the psychopharmacologically acceptable carrier, or excipient such as diluents, preservatives, stabilizers, a wetting agent, an emulsifying agent, a solvent, a sweeting agent, a coloring agent, an osmotic pressure controlling agent, and an antioxidant, general materials may be used according to the dosage form. In the case of formulation, filler, an extending agent, a wetting agent, a disintegrating agent, or surfactant may be used. A representative diluents and excipient may include water, dextrin, calcium carbonate, lactose, propyleneglycol, polyethylene glycol, a vegetable oil such as an olive oil, an injectable ester such as ethyl oleate, liquid paraffin, and normal saline solution.

Specifically, according to the dosage form, in the case of the oral administration, diluents or excipient such as filler, an extending agent, a binding agent, a wetting agent, a disintegrating agent, or surfactant, which is generally used, may be used, while in the case of the parenteral administration, a sterilized aqueous solution, non-aqueous solvent, or suspension, an emulsion, a freeze-dried formulation, suppository, and the like may be used. An example of the non-aqueous solvent or suspension may include propylene glycol, polyethylene glycol, a vegetable oil such as an olive oil, an injectable ester such as ethyl oleate, and the like. An example of such suppository may include Witepsol, Macrogol, Tween 61, Cacao butter, Sevum Laurinum, glycerinated gelatin, or the like.

In addition, the composition for preventing or treating the diseases infected by the methicillin-resistant Staphylococcus aureus according to the present invention may be used in combination with various carriers, such as normal saline solution or an organic solvent, that are acceptable as drugs, and also may be used in combination with carbohydrates such as glucose, sucrose, or dextrin, antioxidants such as ascorbic acid or glutathione, chelating agents, low molecular protein, or other stabilizers in order to increase stability or absorption.

For the composition for preventing or treating the diseases infected by the methicillin-resistant Staphylococcus aureus according to the present invention, an object to be administered may be an echinoderm, crustaceans, mollusks, fish, amphibian, reptile, tidal current, and mammals, preferably, marine organisms including an echinoderm, crustaceans, mollusks, and fish, and mammals that can ingest the marine organisms. More preferably, the marine organisms may be an echinoderm such as spatangoids or holothuroids, crustacean such as crabs, shrimps, and spiny lobster belonging to an arthropod, mollusks such as cephalopods, gastropods, or bivalves, fish, which can be raised trout, such as a red sea-bream, snapper, cod, flat fish, and halibut, and the mammals may be domestic animals such as pigs, cows, and a goat, which can be raised livestock, and fowls such as pheasant or chicken.

For the composition for preventing or treating the diseases infected by the methicillin-resistant Staphylococcus aureus according to the present invention, an dose and use amount may be preferably determined considering a body weight of a patient, age of a patient, sex of a patient, body conditions of a patient, a diet, an administration time, an administration route, excretion, severity of a disease, and the like. For example, an available dose a day may generally be 10 mg/kg to 5000 mg/kg, preferably 100 mg/kg to 3000 mg/kg. It may be administered one time a day or several times a day, and preferably one to three times a day.

Since the dose can be changed according to various conditions, it is understood by those person skilled in the rated art that the dose can be adjusted. Therefore, the range of the present invention is not limited to the dose under any circumstances. The number of administration may be divided into one time a day or several times a day within the desired range and also the administration period is not particularly limited. In addition, the composition for preventing or treating the diseases infected by the methicillin-resistant Staphylococcus aureus according to the present invention may be also routinely ingested by adding the composition to arbitrary foods, in addition to an oral administration.

The composition for preventing or treating the diseases infected by the methicillin-resistant Staphylococcus aureus according to the present invention may provide responds to treatment or prevention of the infection as a result of an excellent antibiotic effect against the methicillin-resistant Staphylococcus aureus, and also is possible to be ingested for a long period time because the composition includes the Eisenia bicyclis extract as an effective component.

Furthermore, the present invention provides a composition for animal feeds, in which the composition includes the antibiotic composition.

Such animals are a living group that corresponds to plants, and often ingests organic matters as a nutrient, of which the digestive organs, the excretion organs, and the respiratory organs are differentiated. A specific example of such animals may include an echinoderm, crustaceans, mollusks, fish, amphibian, reptile, tidal current, and mammals, and preferably, an echinoderm such as spatangoids or holothuroids, an arthropod including crustacean such as crabs, shrimps, and spiny lobster, mollusks such as cephalopods, gastropods, or bivalves, fish such as a red sea-bream, snapper, cod, flat fish, and halibut, fowls including a poultry farm such as pheasant or chicken, a mammal such as human, pigs, cows, and a goat.

The composition for the animal feeds may further include grains, vegetable protein feeds, animality protein feeds, sugars, or milk products in addition to the antibiotic composition including the Eisenia bicyclis extract as an effective component. Specifically, the grains may be ground or broken wheat, oats, barley, corn, and rice; specifically, the vegetable protein feeds may have a rape, beans, and sunflowers as a main component; specifically, the animality protein feeds may be blood meals, meat meals, bone meals, and fish meals; and specifically, the sugar or milk products may be a dried component consisted of all kinds of dry milks and whey powders.

The composition for the animal feeds may be used in combination with components such as nutritional supplements, agents for improving digestive and absorption, a growth promoting agent, or an agent for preventing a disease, supplementally.

The antibiotic composition that is included in the composition for the animal feeds according to the present invention may depend on its use purposes and use conditions, and for example, the antibiotic composition may be included in an amount of 0.1 g to 100 g relative to 1 kg of finally produced feeds.

In addition, the composition for the feeds may be prepared in a granule or a coarse grain having consistency according to the degree of grinding of the components. The composition may be supplied to a mesh, or may be formed in a desired separate form for further processing or packaging. In addition, the composition for the feeds may be subjected to pelletizing, expanding, or extruding process for storage, and excessive water in the composition for the feeds may be preferably dried and removed in order to obtain quality for storage.

In addition, the present invention provides a cleansing agent including the antibiotic composition. The cleansing agent may be preferably a detergent for cloths, dishwashing liquid, a cleansing agent for foods, or a cleansing agent for home appliances. The antibiotic composition is a natural component, and thus harmless to humans. Therefore, most preferably, the cleansing agent according to the present invention is applicable for giving antibiotic action, or cleansing various household items including kitchen utensils or foods.

The cleansing agent may further include additives that are properly selected according to its use purposes and uses, and generally, may be used in combination with other effective components, such as a generally used cleaning agent, or other additives, such as pigments, surfactants, and preservatives. The cleansing agent may be prepared in a powder type, granule type, tablet type, or liquid type according to its use purposes or uses.

In addition, the use amount of the antibiotic composition in the cleansing agent may be properly adjusted according to its use purposes, application types, application sites, an object to be applied, and the like, and for example may be 0.01 parts to 50 parts by weight relative to the whole composition. However, the content of the antibiotic composition is not limited thereto. The antibiotic composition according to the present invention is a natural component, and thus harmless to humans, so that it can be applicable in various amounts as long as the use amount can achieve the application purpose based on the product characteristics.

In addition, the present invention provides a food composition including the antibiotic composition. Such a food composition may include various flavouring agents or natural carbohydrates as a supplement component like a general food composition in addition to the phlorotannin compound derived from Eisenia bicyclis as an effective component.

An example of the above-described natural carbohydrate includes general sugar, such as monosaccharide, for example, glucose, fructose, and the like, disaccharide, for example, maltose, sucrose, and the like, and polysaccharide, for example, dextrin, cyclodextrin, and the like, and sugar alcohol such as xylitol, sorbitol, and erythritol. As the above-described flavouring agents, a natural flavouring agent (Thaumatin), Stevia extract (for example, Rebaudioside A, Glycyrrhizine, and the like), and a synthetic flavouring agent (saccharine, aspartame, and the like) may be advantageously used.

The food composition according to the present invention may be used as functional foods or may be added to various foods by formulating using the same method as the pharmaceutical composition. As foods, in which the composition of the present invention can be included, there are for example, drinks, meats, chocolates, foods, confectionery, pizzas, ramen, other noodles, gums, ice creams, candies, alcohol drinks, vitamins, health supplements, and the like.

In addition, such a food composition may include various nutrients, vitamins, minerals (electrolyte), tastes such synthetic tastes and natural tastes, coloring agents and enhancers (cheese, chocolates, and the like), pectic acid and a salt thereof, alginic acid and a salt thereof, organic acid, protective colloid thickeners, pH adjuster, stabilizers, preservatives, glycerin, alcohols, a carbonating agent for a carbonated drink, and the like, in addition to the extract of bonnemaisonia hamifera heriot, that is an effective component. In addition to that, the food composition of the present invention may include fruit flesh for preparing natural fruit juices, fruit juice drinks, and vegetable drinks.

The health functional foods of the present invention may be prepared and processed in a type of tablets, capsules, powder, liquids, pills, and the like for protecting skin and inhibiting skin damage caused by ultraviolet.

The term, “health functional foods” disclosed in the present invention relates to foods that are prepared and processed by using a raw material or component having useful functionality to humans under the law, 6727, on Health Functional Foods, and means foods that are ingested for controlling nutrients for structures and functions of human body and obtaining a useful effect for health care, such as a physiological effect.

The health function foods of the present invention may include general food additives, and whether or not the health function foods are suitable is determined based on a standard and criteria relating to a relevant item according to general rules disclosed in Korean Food Additives Codex and a general test method as long as other rules do not provide.

The items disclosed in such “Korean Food Additives Codex” may include, for example, a chemically synthetic persimmon color, such as ketones, glycine, calcium citrate, nicotinic acid, and cinnamic acid, a natural additive sodium L-glutamate formulationsuch as licorice extract, microcrystalline cellulose, Kaoliang color, and guar gum, mixed formulations such as alkali agents for noodles, preservative formulation, and tar color formulation.

For example, the health functional foods in a type of tablets may be prepared by granulating the mixture mixing the extract of bonnemaisonia hamifera heriot that is an effective component of the present invention with excipient, a binding agent, a disintegrating agent, and other additives through a general method, and then compression-molding through adding a modifier, and the like, or directly compression-molding the mixture described above. In addition, the health functional foods in a type of tablets may include flavor enhancers, and the like if necessary.

Among the health functional foods in a type of capsules, a hard capsule of the health functional foods may be prepared by filling the mixture mixing the extract of bonnemaisonia hamifera heriot that is an effective component of the present invention with additives such as excipient into a general hard capsule. A soft capsule of the health functional foods may be prepared by filling the mixture mixing the extract of bonnemaisonia hamifera heriot with additives such as excipient into a capsule basic material such as a gelatin. The soft capsule formulation may include a plasticizer such as glycerin or sorbitol, a coloring agent, preservatives, and the like if necessary.

The health functional foods in a type of a pill may be prepared by molding the mixture mixing the extract of bonnemaisonia hamifera heriot that is an effective component of the present invention with excipient, a binding agent, and a disintegrating agent by using a known method in the related art, and if necessary, may be coated with white sugar or other coating agents. In addition, the health functional foods in a type of a pill may be coated with a material such as starch or talc.

The health functional foods in a type of a granule may be prepared by preparing the mixture mixing the extract of bonnemaisonia hamifera heriot that is an effective component of the present invention with excipient, a binding agent, and a disintegrating agent in a type of a granule by using a known method in the related art, and if necessary, may include fragrance ingredients, flavor enhancers, and the like.

The health functional foods may be drinks, meats, chocolates, foods, confectionery, pizzas, ramen, other noodles, gums, ice creams, candies, alcohol drinks, vitamins, health supplements, and the like.

Hereinafter, the present invention will be additionally described with reference to Examples. Examples are only for describing the present invention in more detail, but the range of the present invention is not limited to Examples.

### Example 1

<1-1> Extraction of Eisenia bicyclis Fraction

In the late September 2010, E. bicyclis used for the present invention was purchased from Ulleung Trading Co. to use for the experiment. A voucher specimen is deposited in the food microbiology laboratory in a department of food science and technology at Pukyong National University. E. bicyclis was washed with water to remove salt and then dried. Dried E. bicyclis was ground and then finely powdered with a food mixer (HMF-1000A; Hanil Electronics, Seoul, Korea). The dried powder was stored at −20° C. until required. The dried E. bicyclis powder (1.0 kg) was added with methanol (MeOH; 10 L); then heated at 70° C. for 3 hr in boiling water; and then filtered. Since then, it was decompression-concentrated with a rotary evaporator (Eyela, Tokyo, Japan) to obtain a MeOH extract. Dried MeOH extract (164.3 g) was re-suspended in 1 L H₂O-MeOH (9:1), added with n-hexane (1.0 L), and partitioned in n-hexane extract and water-soluble extract to obtain n-hexane extract. The water-soluble fraction is sequentially added with dichloromethane (DCM), ethyl acetate (EtOAc), and n-butanol (BuOH) to finally obtain dichloromethane, ethyl acetate, n-butanol, and water-soluble extract.

<1-2> Microorganism and Culture

All the bacterial strains used for the Example were obtained from the Korean Collection of Type Cultures (KCTC; Daejeon, Korea) and the Korean Culture Center of Microorganisms (KCCM; Seoul, Korea). The bacterial strains used for the Example were methicillin-susceptible S. aureus (MSSA; KCTC 1927), two MRSA strains (KCCM 40510 and KCCM 40511). All strains were grown in Mueller-Hinton broth (MHB; Difco, Detroit, USA) or Tryptic soy broth (TSB; Difco) for minimum inhibitory concentration (MIC) assay and in Mueller-Hinton agar (MHA; Difco) for disc diffusion.

<1-3> Content of Polyphenol Compound

The content of a polyphenolic compound was evaluated using a Folin-Ciocalteu method. That is, 0.1 mL of 1 mg/mLMeOH extract was added with 0.5 mL of Folin-Ciocalteu reagent, well mixed, and the allowed to stand at room temperature for 3 minutes. After exactingly 3 minutes, 0.4 mL of saturated Na₂CO₃ solution was added, mixed, and allowed to stand at room temperature for 45 minutes. Since then, the mixture was centrifuged for 8 minutes and then the optical density of the supernatant thus obtained was measured at 765 nm.

At this time, the content of the total polyphenol compound was calculated from a calibration curve prepared using a phloroglucinol. The equation of the calibration curve using the phloroglucinol is as follows: y=0.0428x−0.3600.

<1-4> Disk Diffusion Assay

An antibacterial activity was evaluated by a disk diffusion assay. Bacterial strains were cultured in a tryptic soy medium until the cell concentrations reached at about 0.5 of optical density at 600 nm. One mL of bacterial culture containing strains of approximately 10⁴ CFU/mL was spread on a Muller-Hinton agar plate and a paper disc containing 1 mg of each extract was then placed on the plate. After incubating 24 hrs at 37° C., the diameter of inhibition zone was measured.

<1-5> Detection of mecA Gene

In order to detect the mecA gene that encodes penicillin-binding protein 2a (PBP2a) which is intrinsically insensitive to methicillin and to all β-lactams, MSSA and MRSA strain cells were cultured for 18 hrs at 37° C. Then, 3 mL of culture was collected and the chromosomal DNA was prepared using an Accu-Prep Genomic DNA Extraction Kit. The chromosomal DNA was then used as a PCR reaction template. PCR was conducted using two synthetic oligonucleotides as follows:

Sense: 5′-AAAATCGATGGTAAAGGTTGGC-3′;
and
Antisense: 5′-AGTTCTGCAGTACCGGATTTGC-3′.

The DNA was amplified via 30 cycles of denaturation (94° C., 30 s), annealing (50° C., 30 s), and polymerization (72° C., s). The amplified PCR product is anticipated to be approximately 500 bp.

<1-6> Measurement of Minimum Inhibitory Concentrations

MIC means the lowest concentration of antimicrobials that will inhibit the visible growth of microorganisms after overnight incubation. MICS of the extracts and vancomycin were determined by the two-fold serial dilution method in MHB. MIC was defined as the lowest concentration of crude extract that inhibited the visual growth after incubation at 37° C. for 20-24 hrs.

<1-7> Isolation and Purification of Phlorotannins from E. bicyclis

UV spectra were obtained with a Hitachi U-2000 spectrophotometer. The ¹H and ¹³C NMR spectra were measured on a Varian VNS600 instrument operating at 600 and 150 MHz, respectively. The chemical shifts are given in ppm values relative to that of the solvent DMSO-d₆ on a tetramethylsilane (TMS) scale. The standard pulse sequences programmed into the instruments were used for each 2D measurement. The J_{CH} value was set at 8 Hz in the Heteronuclear Multiple Bond Correlation (HMBC) spectra.

Fast Atom Bombardment (FAB-MS) using 3-nitrobenzyl alcohol as the matrix agent was performed on a Micro Mass Auto Spec OA-TOF spectrometer. High-performance liquid chromatography (HPLC) analysis was carried out on a YMC-Pack ODS A-302 column and developed at 40° C. with 1% formic acid (HCOOH:MeCN=8:2; Detection: 280 nm). Column chromatography was performed using LiChroprep RP-18 and Sephadex LH-20. Thin-layer chromatography (TLC) was performed on Kieselgel 60 F₂₅₄ plates and the spots were detected by UV irradiation and by spraying with 10% H₂SO₄ reagent.

<1-8> Analysis of Scanning Electron Microscopy (SEM)

The morphological change of bacterial cells by the treatment of an anti-MRSA substance was monitored using a scanning electron microscopy. The specimens were fixed in 2% phosphate buffered glutaraldehyde, rinsed in 0.05 M phosphate buffer and post-fixed in 1% buffered osmium tetroxide. The fixed samples were then dehydrated stepwise with ethanol. The dried specimens were coated with gold in a SCD-005 sputter-coater and observed using Field Emission Scanning Electron Microscope at an accelerating voltage of 15 kV.

<1-9> Synergic Effects Between Phlorofucofuroeckol-A and β-Lactams Against MRSA

The interaction between phlorofucofuroeckol-A (PFF) and β-lactams including ampicillin, penicillin and oxacillin against MRSA was tested by the checkerboard method. The synergistic effect was evaluated as a fractional inhibitory concentration (FIC) index. The FIC was calculated as the MICS of an antibiotic or phlorofucofuroeckol-A (PFF) in combination divided by the MIC of the antibiotic or PFF alone as follows.

The FIC was then summed to derive the FIC index, which indicated synergy when index values were as follows:

synergism ≦≦0.5, indifference >0.5 to ≦≦4, antagonism >4.

FIC_{A}=MIC_{A} in combination/MIC_{A}, FIC_{B}=MIC_{B} in combination/MIC_{B}

FIC Index=FIC_{A}+FIC_{B}

<1-10> RNA Isolation and RT-PCR Analysis

In order to elucidate the inhibitory effect of phlorofucofuroeckol-A on expression of drug resistance related genes, MRSA cells were treated with various concentrations of phlorofucofuroeckol-A. After cell harvesting, Total RNA was isolated from the strains with zirconia beads and RNAwiz with the manufacturer's specifications. RNA concentrations were estimated via spectrophotometer at 260 nm. The 0.2-1.4 μg of total RNA plus 1.4 μg of random primer was denatured at 65° C. for 5 min, then cooled at 30 sec and preincubated for 2 min at 37° C. after the addition of 10 mM dithioothreitol (DTT), 2.5 mM each of dNTPs, and reaction buffer. Any remaining cRNA was removed via the addition of 2 units RNase H at 37° C. for 20 min. One hundred units of Superscript II reverse transcriptase were added and incubated for 50 min at 37° C. The reaction was then suspended at 70° C. for 15 min. One point five percent of the RT products were added to a PCR reaction which included PCR buffer (pH 8.4, 20 mM Tris, 50 mM KCl), 1.5 mM MgCl₂, 0.5 mM dNTPs, 2 pM primers and 0.1 μL Taq DNA polymerase. Twenty-eight PCR cycles were then conducted follows: denaturation at 95° C. annealing at an indicated temperature for each primer and extension at 72° C.

Primer sequences were as follows: mecA (554 bp, PCR product, annealing temperature: 51.9° C.) F; 5′-ATGAGATTAGGCATCGTTCC-3′, R; 5′-TGGATGACAGTACCTGAGCC-3′; mecI (268 bp PCR product, annealing temperature: 49.5° C.) F; 5′-CTGCAGAATGGGAAGTTATG-3′, R; 5′-ACAAGTGAATTGAAACCGCC-3′; mecRI (235 bp PCR product, annealing temperature: 53.9° C.) F; 5′-AAGCACCGTTACTATCTGCACA-3′, R; 5′-GAGTAAATTTTGGTCGAATGCC-3′; femA (372 bp PCR product, annealing temperature: 52.6° C.) F; 5′-CATGATGGCGAGATTACAGGCC-3′, R; 5′-CGCTAAAGGTACTAACACACGG-3′; GAPDH (514 bp PCR product, annealing temperature: 51.0° C.) F; 5′-ATGACCCCTTCATTGACC-3′, R; 5′-GAAGATGGTGATGGGATTTC-3′ (Lee et al., 2007; Lei et al., 2007).

<1-11> Western Blot Analysis

In order to elucidate the inhibitory effect of phlorofucofuroeckol-A on expression of a drug resistance related protein, PBP2a, MRSA cells were treated with various concentrations of phlorofucofuroeckol-A. After cell harvesting, the bacterial lysates were prepared in a lysis buffer containing 20 mM Tris-HCl (pH 7.5), 2 mM ethyleneglycoltetraacetic acid (EGTA), 2 mM ethylenediaminetetraacetic acid (EDTA) and 0.25 M sucrose. The pellets were resuspended by sonication in lysis buffer 2 times for 20 sec. Following 10 min of centrifugation at 13,000×g, the supernatant was obtained as the cell lysate. Protein concentrations were measured with Bradford protein assay. An equal amount of 2×SDS-PAGE sample buffer (pH 7.5, mM Tris-HCl, 1 mM EGTA, 1 mM EDTA, 1% SDS, 150 mM NaCl) was added to the tubes containing the cell lysate and the tubes were boiled for 3 min. Aliquots of cellular proteins were then electrophoresed on 10%.

Statistical Analysis

In all cases, analyses were performed in triplicate and data were averaged over the three measurements. The standard deviation was also calculated. Data were treated for multiple comparisons by analysis of variance (ANOVA). ANOVA was performed using the statistical software SPSS version 12.0. Significant differences between means were determined by using Duncan's Multiple Range tests. Significance of differences was defined at the P<0.05 level.

### Experimental Example 1

Sequential Fractions of E. bicyclis Extracts

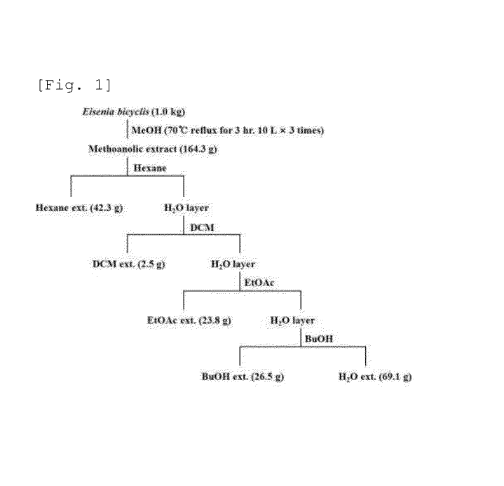

E. bicyclis extracts were sequentially fractionated and then weight of the fractions thus obtained were arranged (see FIG. 1). A lyophilized powder of E. bicyclis was percolated in methanol (3 times×1.0 L), followed by partitioning with several organic solvents to yield Hexane-soluble extract (42.3 g), DCM-soluble extract (2.5 g), EtOAc-soluble extract (23.8 g), BuOH-soluble extract (26.5 g) and H₂O-soluble extract (69.1 g) in extract.

### Experimental Example 2

TP Contents of the MeOH Extract and its Solvent Extracts

The total polyphenol content of the solvent extracts are shown in the following Table 1. Among E. bicyclis extracts, the TP content of EtOAC-soluble extract was the highest (739.2 mg PGEs/g of dry basis) followed by DCM-soluble extract (394.2 PGEs/g of dry basis), BuOH-soluble extract (247.7 mg PGEs/g of dry basis), Hexane-soluble extract (56.1 PGEs/g of dry basis) and H2O-soluble extract (52.9 PGEs/g of dry basis). These results revealed that the order of the amounts of total polyphenol was similar to that of their antimicrobial activities as shown in the following Table 2. Therefore, it was considered that antimicrobial activities of E. bicyclis extracts also correlated with their polyphenol contents.

TABLE 1
Total polyphenol content expressed as
phloroglucinol equivalents in Eisenia bicyclis extracts

| | Total phenolics* |
|---|---|
| Samples^{†} | (mg PGE/g, dry basis) |
| MeOH | 85.3 ± 18.5^{b} |
| Hexane | 56.1 ± 4.8^{a} |
| DCM | 394.2 ± 6.2^{d} |
| EtOAc | 739.2 ± 24.2^{e} |
| BuOH | 247.7 ± 18.2^{b} |
| H₂O | 52.9 ± 0 9.1^{a} |

^{†}MeOH, methanolic extract; DCM, dichloromethane-soluble extract; EtOAc, ethyl acetate-soluble extract; BuOH, butanol-soluble extract; H₂O, water-soluble extract
*Data are the means of three replications ± standard deviation.

### Experimental Example 3

Anti-MRSA Activity Analysis of E. bicyclis Extracts

The anti-MRSA activities of the E. bicyclic extracts and the soluble fractions were measured though a disk diffusion assay. Among them, the EtOAc-soluble extract showed the strongest anti-MRSA activity and followed by DCM-, BuOH- and Hexane-soluble extract in the order (see Table 1 and Table 2). No anti-MRSA activity was observed in water-soluble extract.

TABLE 2
Disk diffusion assay of E. bicyclis extracts against
methicillin-resistant Staphylococcus aureus

Gram-

| positive | | Zone of inhibition (mm)^{a} | | | | | |
|---|---|---|---|---|---|---|---|
| bacteria | Concn. | MeOH^{a} | Hexane | DCM | EtOAc | BuOH | H₂O |
| MSSA | 1 mg/ | 10.0 ± | 10.5 ± | 10.0 ± | 15.0 ± | 9.5 ± | —^{d} |
| (KCTC | disk^{b} | 0.2^{c} | 0.3 | 0.2 | 0.4 | 0.3 | |
| 1927) | 5 mg/ | 16.5 ± | 10.0 ± | 13.5 ± | 19.0 ± | 12.0 ± | — |
| | disk | 0.3 | 0.2 | 0.2 | 0.3 | 0.2 | |
| MRSA | 1 mg/ | 13.5 ± | 10.0 ± | 15.5 ± | 17.0 ± | 10.0 ± | — |
| (KCCM | disk | 0.4 | 0.1 | 0. 1 | 0.4 | 0.1 | |
| 40510) | 5 mg/ | 18.0 ± | 17.5 ± | 22 .0 ± | 24.0 ± | 15.0 ± | — |
| | disk | 0.1 | 0.3 | 0.4 | 0.6 | 0.1 | |
| MRSA | 1 mg/ | 11.0 ± | 9.0 ± | 11.0 ± | 16.0 ± | 10.0 ± | — |
| (KCCM | disk | 0.2 | 0.2 | 0.1 | 0.2 | 0.2 | |
| 40511) | 5 mg/ | 18.0 ± | 11.0 ± | 14 .0 ± | 21.0 ± | 14.0 ± | — |
| | disk | 0.2 | 0.3 | 0.5 | 0.3 | 0.1 | |

^{a}MeOH, methanolic extract; DCM, dichloromethane-soluble extract; EtOAc, ethyl acetate-soluble extract; BuOH, butanol-soluble extract; H₂O, water-soluble extract
^{b}Methanol extract and its fractionated extract from Eisenia bicyclis was loaded onto a disk (6 mm in diameter).
^{c}Data are the averages of duplicate experiments.
^{d}no detected antibacterial activity.

### Experimental Example 4

Determination of MIC of E. bicyclis Extracts

In order to quantitatively evaluate its antibacterial activity against MRSA, MIC values of the extract against the pathogenic bacteria including MSSA and MRSA were investigated. Table 3 shows minimum inhibitory concentrations of E. bicyclic extracts against photogenic bacteria including MSSA and MRSA. The highest anti-MRSA activity was observed on the EtOAc-soluble extract. These results were also consisted with the results obtained by the disk diffusion assay. These results strongly suggested that an anti-MRSA substance originated from the E. bicyclis methanolic extract will be abundant in the EtOAc-soluble extract.

TABLE 3
Minimum inhibitory concentrations of Eisenia bicyclis
extracts against methicillin-resistant

| | MIC (μg/mL) | | | | | | |
|---|---|---|---|---|---|---|---|
| | | | | | | | Vanco- |
| Strains | MeOH* | Hexane | DCM | EtOAc | BuOH | H2O | mycin |
| MSSA | 128 | 128 | 64 | 32 | 128 | >512 | 0.5 |
| (KCTC | | | | | | | |
| 1927) | | | | | | | |
| MRSA | 64 | 64 | 128 | 32 | 128 | >512 | 2 |
| (KCCM | | | | | | | |
| 40510) | | | | | | | |
| MRSA | 128 | 128 | 128 | 64 | 128 | >512 | 2 |
| (KCCM | | | | | | | |
| 40511) | | | | | | | |

Staphylococcus aureus
*MeOH, methanolic extract; DCM, dichloromethane-soluble extract; EtOAc, ethyl acetate-soluble extract; BuOH, butanol-soluble extract; H₂O, water-soluble extract

### Experimental Example 5

Isolation of Phlorotannins from E. bicyclis

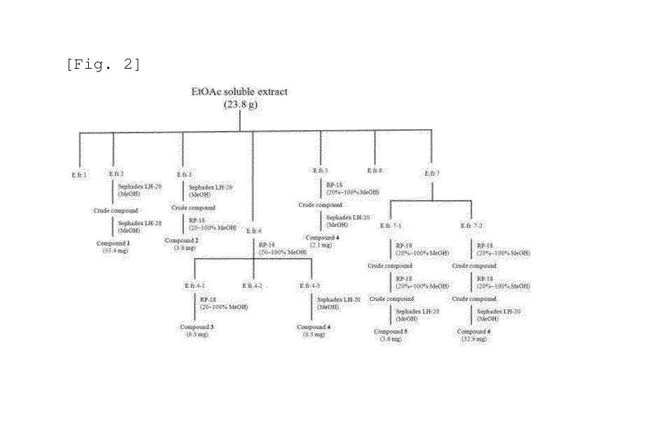

The EtOAc-soluble extract of E. bicyclis solvent showing the highest anti-MRSA activity in vitro was subjected to isolate active compounds. First, the EtOAc-soluble extract (23.8 g) was chromatographed on a Sephadex LH-20 column using MeOH as solvent to yield 7 subfractions (FIG. 2). Subfractions EF02 and EF03 were subjected to column chromatography over a LiChroprep RP-18 column (1.1 cm i.d.×37 cm) with aqueous MeOH to yield pure compounds 1 (55.4 mg) and 2 (3.6 mg). Subfractions EF04 and EF05 were subjected to Sephadex LH-20 column (1.1 cm i.d.×38 cm) chromatography and LiChroprep RP-18 (1.1 cm i.d.×37 cm) with aqueous MeOH to yield pure compounds 3 (6.3 mg) and 4 (5.6 mg). Similarly, subfraction EF07 was chromatographed over Sephadex LH-20 column (1.1 cm i.d.×38 cm) chromatography and LiChroprep RP-18 (1.1 cm i.d.×37 cm) with aqueous MeOH to yield pure compounds 5 (3.6 mg) and 6 (32.9 mg).

### Experimental Example 6

Identification of Compounds Isolated from E. bicyclis

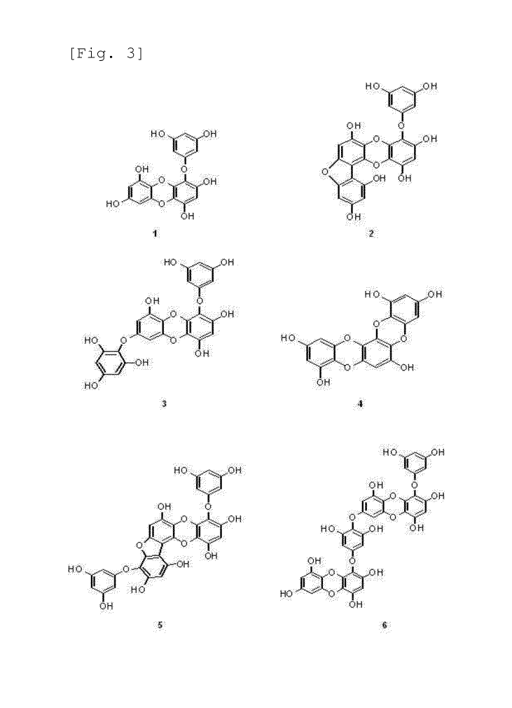

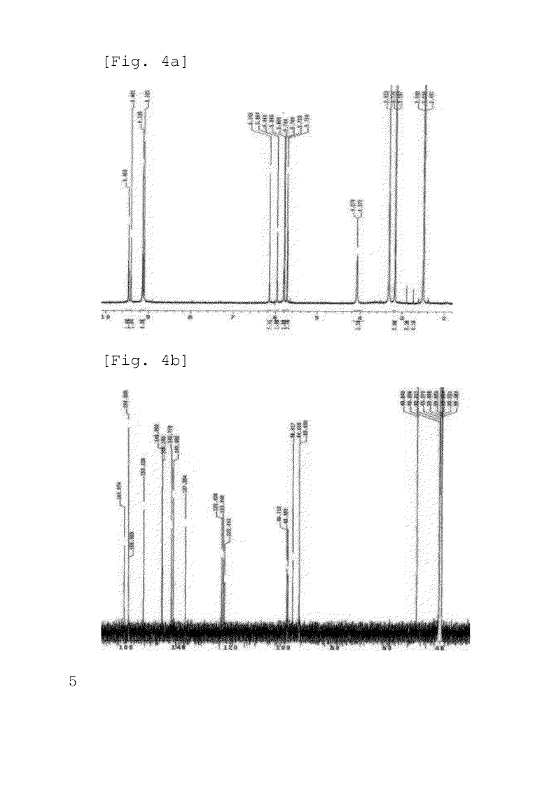

In Experimental Example 5, successive chromatographic purification of the EtOAc-soluble extract led to the isolation and characterization of six phloroglucinol derivatives as follows: compound 1 is eckol, compound 2 is fucofuroeckol A, compound 3 is 7-phloroeckol, compound 4 is dioxinodehydroeckol, compound 5 is phlorofucofuroeckol-A, and compound 6 is dieckol (see FIG. 3). In addition, FIGS. 4A to 9B are the results of ¹H-NMR spectrum (A) and ¹³C-NMR spectrum (B), and the identification results of phlorotannins isolated through FAB-MS and HPLC are as follows.

Compound 1 (eckol, EK): pale brown powder, FAB-MS m/z 373 [M+H]⁺. C₁₈H₁₂O₉. ¹H-NMR (DMSO-d₆, 600 MHz) δ: 9.46 (1H, s, OH-9), 9.41 (1H, s, OH-4), 9.14 (2H, s, OH-2, 7), 9.11 (2H, s, OH-3′, 5′), 6.14 (1H, s, H-3), 5.96 (1H, d, J=2.4 Hz, H-8), 5.80 (1H, d, J=1.8 Hz, H-6), 5.79 (1H, d, J=3.0 Hz, H-4′), 5.72 (2H, d, J=1.8 Hz, H-2′, 6′). ¹³C-NMR (DMSO-d₆, 100 MHz) δ: 160.6 (C-1′), 159.0 (C-3′, 5′), 153.2 (C-7), 146.3 (C-9), 146.1 (C-2), 142.8 (C-5a), 142.1 (C-4), 137.4 (C-10a), 123.4 (C-1), 122.9 (C-9a), 122.5 (C-4a), 98.7 (C-8), 98.4 (C-3), 96.4 (C-4′), 93.9 (C-2′), 93.8 (C-6), 93.7 (C-6′); see Table 4 and FIGS. 4A and 4B.

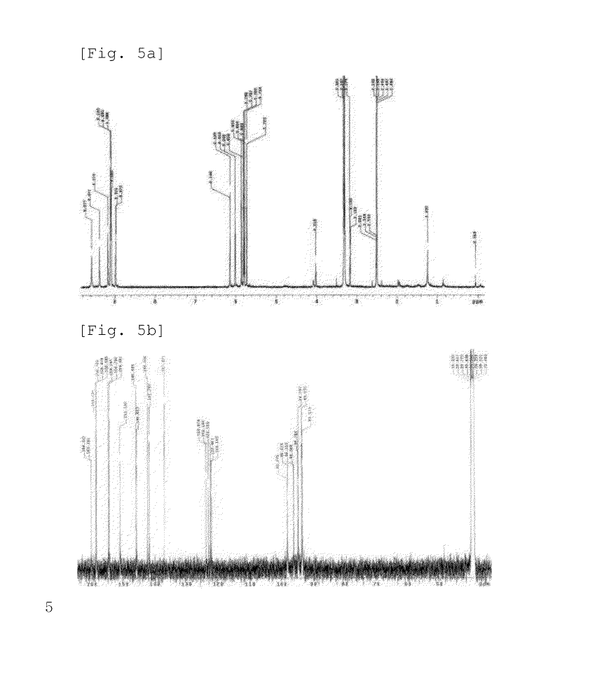

Compound 2 (fucofuroeckol-A, FFA): pale brown powder, FAB-MS m/z 479 [M+H]⁺. C₂₂H₁₄O₁₁. ¹H NMR (DMSO-d₆, 600 MHz) δ: 10.05 (1H, s, OH-14), 9.85 (1H, s, OH-4), 9.73 (1H, s, OH-10), 9.44 (1H, s, OH-2), 9.14 (2H, s, 3′,5′-OH), 8.21 (1H, s, 8-OH), 6.72 (1H, s, H-13), 6.47 (1H, d, J=2.4 Hz, H-11), 6.29 (1H, s, H-3), 6.25 (1H, d, J=1.2 Hz, H-9), 5.83 (1H, d, J=2.4 Hz, H-4′), 5.77 (2H, d, J=2.4 Hz, H-2′, 6′). ¹³C-NMR (DMSO-d₆, 100 MHz) δ: 160.2 (C-1′), 158.8 (C-3′, 5′), 158.2 (C-11a), 157.6 (C-10), 150.4 (C-12a), 150.2 (C-8), 146.8 (C-2), 144.3 (C-14), 141.9 (C-4), 136.8 (C-15a), 133.6 (C-5a), 126.1 (C-14a), 122.6 (C-4a), 122.4 (C-1), 103.1 (C-6), 102.4 (C-7), 98.2 (C-3), 98.0 (C-9), 96.3 (C-4′), 94.6 (C-13), 93.7 (C-2′, 6′), 90.5 (C-11); see Table 4 and FIGS. 5A and 5B.

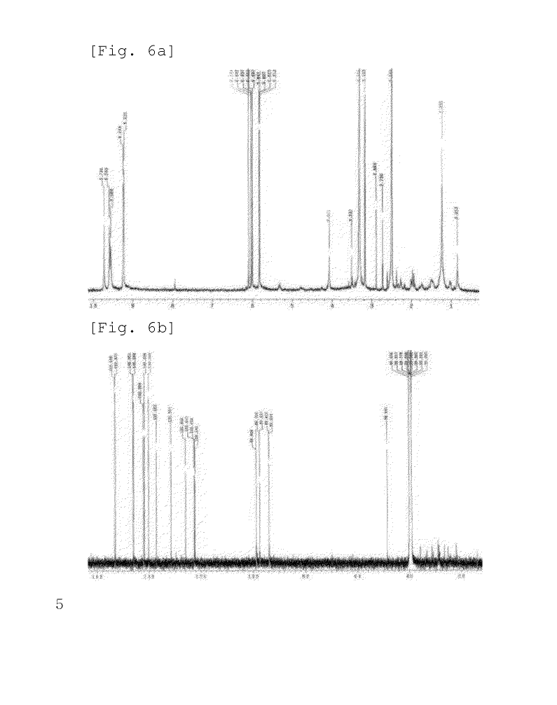

Compound 3 (1-(3′,5′-dihydroxyphenoxy)-7-(2″,4″,6-trihydroxyphenoxy)-2,4,9-trihydroxydibenzo-1,4-dioxin, 7-phloroeckol, 7-P): light-brown powder, FAB-MS m/z 497 [M+H]⁺. C₂₄H₁₆O₁₂. ¹H NMR (DMSO-d₆, 600 MHz) δ: 9.56 (1H, s, OH-9), 9.38 (1H, s, OH-4), 9.17 (1H, s, OH-2), 9.10 (2H, s, OH-3′, 5′), 9.09 (1H, s, OH-2″), 9.08 (1H, s, OH-6″), 8.98 (1H, s, OH-4″), 6.14 (1H, s, H-3), 6.01 (1H, d, J=2.4 Hz, H-8), 5.86 (2H, s, H-3″, 5″), 5.80 (1H, t, J=2.4 Hz, H-4′), 5.79 (1H, d, J=2.4 Hz, H-6), 5.73 (2H, d, J=1.8 Hz, H-2′, 6′). ¹³C-NMR (DMSO-d₆, 100 MHz) δ: 160.2 (C-1′), 158.7 (C-3′,5′), 154.8 (C-6″), 154.7 (C-4″), 154.5 (C-7), 151.3 (C-2″), 145.9 (C-9), 145.8 (C-2), 142.3 (C-5a), 141.8 (C-4), 137.0 (C-10a), 123.9 (C-9a), 123.1 (C-4a), 122.5 (C-1″), 122.2 (C-1), 98.3 (C-3), 98.1 (C-8), 96.2 (C-4′), 94.8 (C-3″), 94.7 (C-5″), 93.6 (C-2′, 6′), 93.4 (C-6); see Table 4 and FIGS. 6A and 6B.

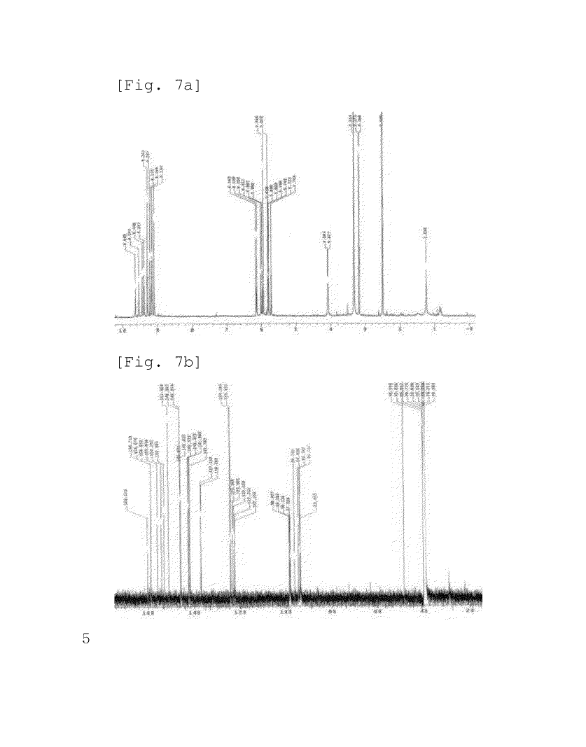

Compound 4 (dioxinodehydroeckol, DD): pale brown powder, FAB-MS m/z 371 [M+H]⁺. C₁₈H₁₀O₉. ¹H-NMR (DMSO-d₆, 600 MHz) δ: 9.73 (1H, s, OH-1), 9.59 (1H, s, OH-9), 9.56 (1H, s, OH-6), 9.24 (1H, s, OH-3), 9.23 (1H, s, OH-11), 6.10 (1H, s, H-7), 6.04 (1H, d, J=2.7 Hz, H-2), 6.01 (1H, d, J=2.7 Hz, H-10), 5.84 (1H, d, J=2.7 Hz, H-4), 5.82 (1H, d, J=2.7 Hz, H-12). ¹³C-NMR (DMSO-d₆, 100 MHz) δ: 153.3 (C-3), 153.0 (C-11), 146.3 (C-1), 146.1 (C-9), 142.1 (C-4a), 141.7 (C-12a), 140.1 (C-6), 137.2 (C-7a), 131.6 (C-13b), 125.9 (C-5a), 122.6 (C-8a), 122.4 (C-13a), 122.2 (C-14a), 98.8 (C-2, 10), 97.5 (C-7), 93.9 (C-4, 12); see Table 4 and FIGS. 7A and 7B.

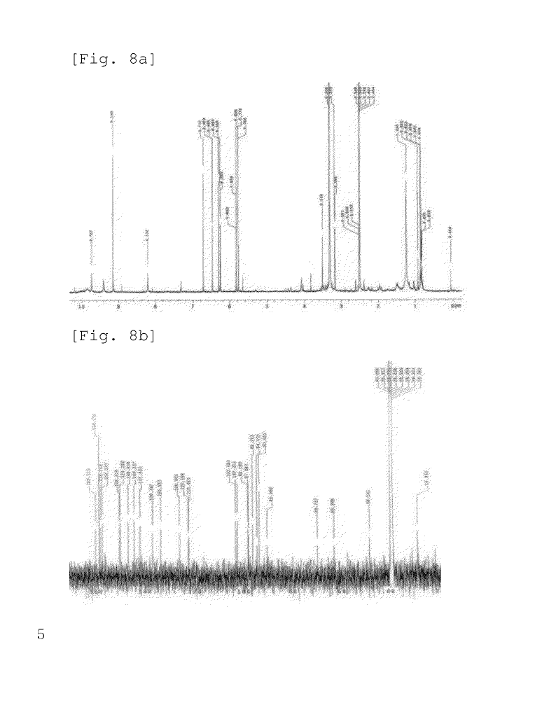

Compound 5 (phlorofucofuroeckol A, PFF): light-brown powder, FAB-MS m/z 603 [M+H]⁺. C₃₀H₁₈O₁₄. ¹H-NMR (DMSO-d₆, 600 MHz) δ: 10.12 (1H, s, OH-14), 9.85 (1H, s, OH-4), 9.42 (1H, s, OH-10), 9.25 (1H, s, OH-2), 9.18 (2H, s, OH-3″,5″), 9.15 (2H, s, OH-3′, 5′), 8.20 (1H, s, OH-8), 6.72 (1H, s, H-13), 6.42 (1H, s, H-9), 6.30 (1H, s, H-3), 5.83 (2H, t, J=2.4 Hz, H-4′, 4″), 5.77 (2H, d, J=1.8 Hz, H-2′, 6′), 5.73 (2H, d, J=1.8 Hz, H-2″, 6″). ¹³C-NMR (DMSO-d₆, 100 MHz) δ: 159.8 (C-1′), 159.5 (C-1″), 158.6 (C-3″, 5″), 158.4 (C-3′, 5′), 150.4 (C-10), 149.9 (C-12a), 149.1 (C-11a), 146.5 (C-2), 146.1 (C-8), 144.3 (C-14), 141.6 (C-4), 136.4 (C-15a), 133.5 (C-5a), 125.9 (C-14a), 122.2 (C-1, 4a), 119.7 (C-11), 103.0 (C-7), 102.8 (C-6), 98.7 (C-9), 97.8 (C-3), 96.1 (C-4″), 95.9 (C-4′), 94.4 (C-13), 93.3 (C-2′), 93.3 (C-6′), 93.1 (C-2″, 6″); see Table 4 and FIGS. 8A and 8B.

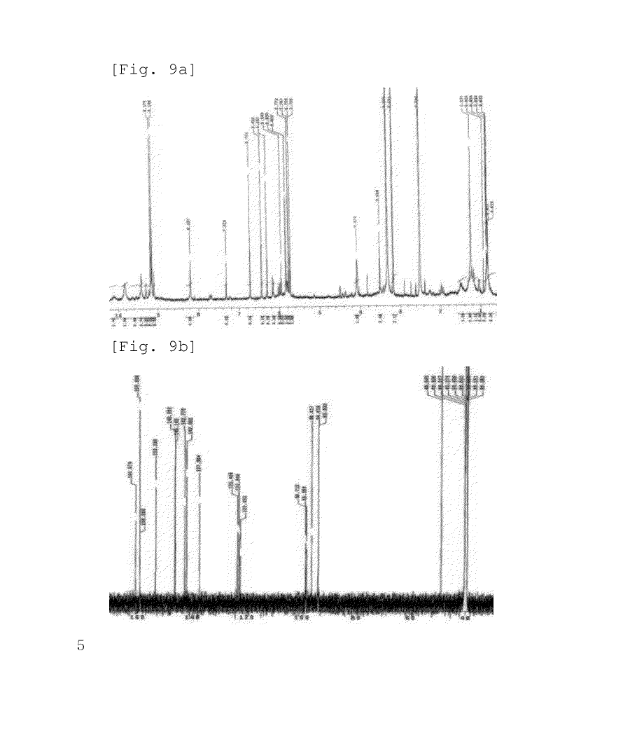

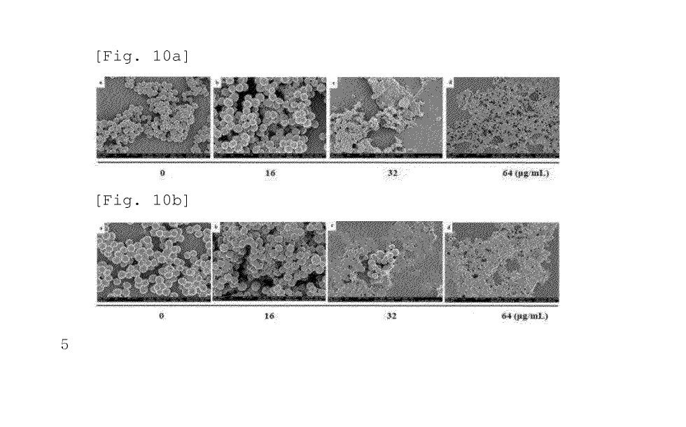

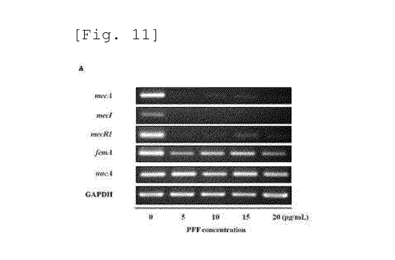

Compound 6 (dieckol, DE): pale brown powder, FAB-MS m/z 743 [M+H]⁺. C₃₆H₂₂O₁₈. ¹H-NMR (DMSO-d₆, 600 MHz) δ: 9.65 (1H, s, OH-9), 9.55 (1H, s, OH-9″), 9.45 (1H, s, OH-4″), 9.40 (1H, s, OH-4), 9.31 (2H, s, OH-3″, 5″), 9.23 (1H, s, OH-2″), 9.18 (1H, s, OH-2), 9.17 (1H, s, OH-7″), 9.10 (2H, s, OH-3′, 5′), 6.16 (1H, s, H-3″), 6.14 (1H, s, H-3), 6.02 (1H, d, J=3.0 Hz, H-8), 5.99 (1H, d, J=3.0 Hz, H-8″), 5.95 (2H, s, H-2″, 6″), 5.82 (1H, d, J=3.0 Hz, H-6), 5.81 (1H, d, J=3.0 Hz, H-6″), 5.80 (1H, d, J=1.8 Hz, H-4′), 5.72 (2H, d, J=1.8 Hz, H-2′, 6′). ¹³C-NMR (DMSO-d₆, 100 MHz) δ: 160.2 (C-1′), 158.7 (C-3′) 158.6 (C-5′), 155.8 (C-1″), 154.2 (C-7), 153.0 (C-7″), 151.1 (C-3″, 5″), 146.0 (C-2, 9″), 145.8 (C-2″, 9), 142.5 (C-5a″), 142.3 (C-5a), 141.9 (C-4″), 141.8 (C-4), 137.2 (C-10a), 137.0 (C-10a″), 124.2 (C-4″), 124.0 (C-9a), 123.2 (C-4a), 123.1 (C-4a″), 122.6 (C-9a″), 122.2 (C-1, 1″), 98.3 (C-3), 98.2 (C-3″), 98.0 (C-8, 8″), 96.1 (C-4′), 94.4 (C-2″, 6″), 93.8 (C-6″), 93.6 (C-2′, 6′), 93.5 (C-6); see Table 4 and FIGS. 9A and 9B.

### Experimental Example 7

Spectroscopic Characteristics of Compounds 1 to 6 Isolated from EtOHc-Soluble Extracts

The following Table 4 is NMR values and data of compounds 1 to 6 isolated from EtOHc-soluble extracts.

TABLE 4
¹³C NMR (600 MHz) data for isolated
phlorotannins (1-6) in DMSO-d₆

| | Com- | Com- | Com- | Com- | Com- | Com- |
|---|---|---|---|---|---|---|
| Position | pund 1 | pound 2 | pound 3 | pound 4 | pound 5 | pound 6 |
| 1 | 123.4 | 122.4 | 122.2 | 146.3 | 122.2 | 122.2 |
| 2 | 146.1 | 146.8 | 145.8 | 98.8 | 146.5 | 146.0 |
| 3 | 98.4 | 98.2 | 98.3 | 153.3 | 97.8 | 98.3 |
| 4 | 142.1 | 141.9 | 141.8 | 93.9 | 141.6 | 141.8 |
| 4a | 122.5 | 122.6 | 123.1 | 142.1 | 122.2 | 123.2 |
| 5a | 142.8 | 133.6 | 142.3 | 125.9 | 133.5 | 142.3 |
| 6 | 93.8 | 103.1 | 93.4 | 140.1 | 102.8 | 93.5 |
| 7 | 153.2 | 102.4 | 154.5 | 97.5 | 103.0 | 154.2 |
| 7a | | | | 137.2 | | |
| 8 | 98.7 | 150.2 | 98.1 | | 146.1 | 98.0 |
| 8a | | | | 122.6 | | |
| 9 | 146.3 | 98.0 | 145.9 | 146.1 | 98.7 | 145.8 |
| 9a | 122.9 | | 123.9 | | | 124.0 |
| 10 | | 157.6 | | 98.8 | 150.4 | |
| 10a | 137.4 | | 137.0 | | | 137.2 |
| 11 | | 90.5 | | 153.0 | 119.7 | |
| 11a | | 158.2 | | | 149.1 | |
| 12 | | | | 93.9 | | |
| 12a | | 150.4 | | 141.7 | 149.9 | |
| 13 | | 94.6 | | | 94.4 | |
| 13a | | | | 122.4 | | |
| 13b | | | | 131.6 | | |
| 14 | | 144.3 | | | 144.3 | |
| 14a | | 126.1 | | 122.2 | 125.9 | |
| 15a | | 136.8 | | | 136.4 | |
| 1′ | 160.6 | 160.2 | 160.2 | | 159.8 | 160.2 |
| 2′ | 93.9 | 93.7 | 93.6 | | 93.3 | 93.6 |
| 3′ | 159.0 | 158.8 | 158.7 | | 158.4 | 158.7 |
| 4′ | 96.4 | 96.3 | 96.2 | | 95.9 | 96.1 |
| 5′ | 159.0 | 158.8 | 158.7 | | 158.4 | 158.6 |
| 6′ | 93.7 | 93.7 | 93.6 | | 93.3 | 93.6 |
| 1″ | | | 122.5 | | 159.5 | 122.2 |
| 2″ | | | 151.3 | | 93.1 | 145.8 |
| 3″ | | | 94.8 | | 158.6 | 98.2 |
| 4″ | | | 154.7 | | 96.1 | 141.9 |
| 4a″ | | | | | | 123.1 |
| 5″ | | | 94.7 | | 158.6 | |
| 5a″ | | | | | | 142.5 |
| 6″ | | | 154.8 | | 93.1 | 93.8 |
| 7″ | | | | | | 153.0 |
| 8″ | | | | | | 98.0 |
| 9″ | | | | | | 146.0 |
| 9a″ | | | | | | 122.6 |
| 10a″ | | | | | | 137.0 |
| 1′″ | | | | | | 155.8 |
| 2′″ | | | | | | 94.4 |
| 3′″ | | | | | | 151.1 |
| 4′″ | | | | | | 124.2 |
| 5′″ | | | | | | 151.1 |
| 6′″ | | | | | | 94.4 |

| | | MIC (μg/mL) | | | |
|---|---|---|---|---|---|
| Strains | mecA ^{a} | PFF | Ampicillin | Penicillin | Oxacillin |
| MSSA | − | 32 | 0.063 | 0.125 | 0.063 |
| (KCTC 1927) | | | | | |
| MRSA | + | 32 | 512 | 512 | 256 |
| (KCCM40510) | | | | | |
| MRSA | + | 32 | 512 | 256 | 128 |
| (KCCM40511) | | | | | |

### Experimental Example 8

MIC Values of Phlorotanins Derived from E. bicyclis Against MRSA

Table 5 is MIC results of phlorofucofuroeckol-A and β-lactams and Table 6 is MIC results of phlorotannins isolated from Eisenia bicyclis.

[Table 5]

Minimum inhibitory concentration of phlorofucofuroeckol-A and β-lactams against methicillin-susceptible Staphylococcus aureus and methicillin-resistant S. aureus

^{a}+, mecA positive; −, mecA negative.

TABLE 6
Minimum inhibitory concentration of phlorotannins isolated from
Eisenia bicyclis against methicillin-susceptible Staphylococcus aureus
and methicillin-resistant S. aureus

| | MIC (μg/mL)^{a} | | | | | |
|---|---|---|---|---|---|---|
| Strains | EK | FF | PE | DD | PFF | DE |
| MSSA (KCTC 1927) | 64 | 32 | 64 | 64 | 32 | 32 |
| MRSA (KCCM40510) | 64 | 64 | 32 | 64 | 32 | 32 |
| MRSA (KCCM40511) | 64 | 64 | 64 | 64 | 32 | 64 |

EK, eckol; FF, fucofuroeckol-A; PE, 7-phloroeckol; DD, dioxinodehydroeckol; PFF, phlorofucofuroeckol-A; DE, dieckol.

### Experimental Example 9

Synergistic Effects Between PFF and β-Lactams Against MSSA and MRSA

The following Table 7 is results about synergistic effects of phlorofucofuroeckol-A and β-lactams antibiotics having high anti-bacteria activity relatively among phlorotannins isolated against MSAA and MRSA.

TABLE 7
Minimum inhibitory concentrations and fractional inhibitory concentration indices of phlorofucofuroeckol-A
in combination with (β-lactams against methicillin-resistant Staphylococcus aureus

| | Ampicillin | | | | | Penicillin | | | | | Oxacillin | | | | |
|---|---|---|---|---|---|---|---|---|---|---|---|---|---|---|---|
| | MIC (μg/mL) | | | FIC index | | MIC (μg/mL) | | | FIC index | | MIC (μg/mL) | | | FIC index | |
| Strain | A | B | C | b | c | A | B | C | b | c | A | B | C | b | c |
| MSSA | 0.063 | 0.063 | 0.063 | 1.391 | 1.781 | 0.125 | 0.125 | 0.125 | 1.391 | 1.781 | 0.063 | 0.063 | 0.063 | 1.391 | 1.781 |
| (KCCTC | | | | | | | | | | | | | | | |
| 1927) | | | | | | | | | | | | | | | |
| MRSA | 512 | 16 | 8 | 0.422 | 0.407 | 512 | 4 | 2 | 0.399 | 0.395 | 256 | 8 | 4 | 0.422 | 0.407 |
| (KCCM | | | | | | | | | | | | | | | |
| 40510) | | | | | | | | | | | | | | | |
| MRSA | 512 | 2 | 1 | 0.395 | 0.393 | 256 | 1 | 0.5 | 0.395 | 0.393 | 128 | 1 | 0.5 | 0.399 | 0.395 |
| (KCCM | | | | | | | | | | | | | | | |
| 40511) | | | | | | | | | | | | | | | |

A, without phlorofucofuroeckol-A; B to C and b to c, phlorofucofuroeckol-A at 12.5 and 25 μg/ml, respectively.
^{a)} The FIC index indicated synergism ≦0.5, indifference >0.5 to ≦4, antagonism >4.

As shown in the above-described results, PFF (25 pg/mL) can remarkably reduce the MICS of the β-lactams against MRSA. That is, this finding indicates that the β-lactam antibiotics could restore antibacterial activity against MRSA in the presence of PFF. These values are less than that of vancomycin (2 μg/mL) against MRSA, suggesting that PFF may have potential for developing a drug for the treatment, such as a phytotherapeutic for controlling antibiotic-resistant bacteria.

### Experimental Example 10

Effect of PFF on MRSA Cell Morphology

FIG. 10 is scanning electron microscopic profiles on the antimicrobial effect of phlorofucofuroeckol-A against methicillin-resistant Staphylococcus aureus. In FIG. 10, (A) KCCM 40510 (B) KCCM 40511. (a) Control, x30,000 magnification (b) treated with phlorofucofuroeckol-A of 16 μg/mL (c) treated with phlorofucofuroeckol-A of 32 μg/mL (d) treated with phlorofucofuroeckol-A of 64 μg/mL.

### Experimental Example 11

Inhibitory Activity of PFF on the Expression of Genes and the Production of PBP2a Related in Drug Resistance

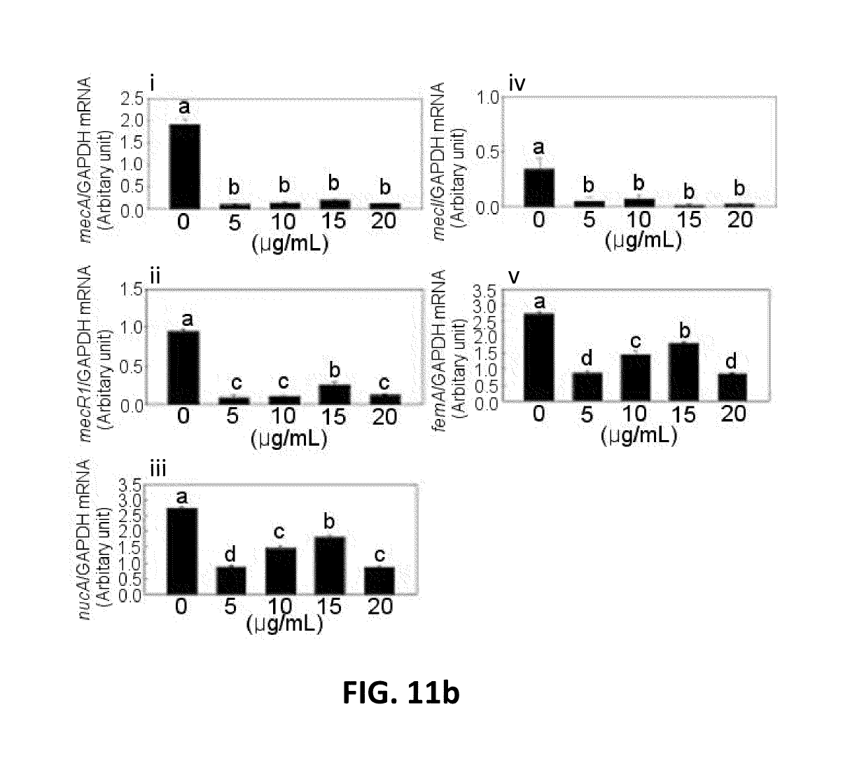

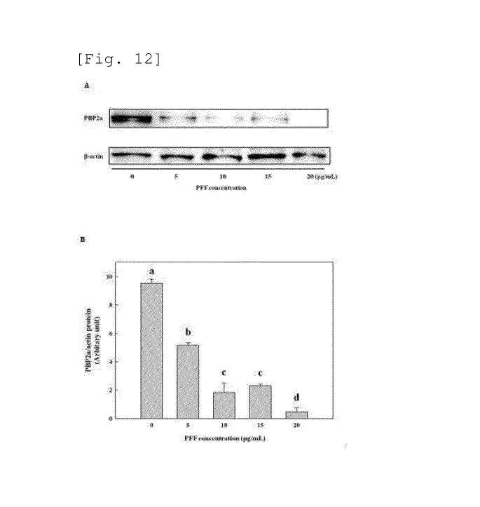

FIGS. 11 and 12 are results of confirming an effect of PFF on PBP2a protein expression relating to gene transcription and drug-resistance using a RT-PCR and a western blot. It was observed that the phlorofucofuroeckol-A inhibited the gene transcription of mec operon (mecA, mecI, mecR1) involving in synthesizing PBP2a depending on the concentrations. In addition, it was confirmed from a result of the western blot that the phlorofucofuroeckol-A inhibited a PBP2a synthesis that is a final product of mecA gene depending on the concentrations. From such results, the conclusion was deducted as follows: the phlorofucofuroeckol-A recovers the antibiotics sensitivity against β-lactam-based antibiotics by inhibiting a synthesis of PBP2a protein that directly inhibits the transportation of β-lactam-based antibiotics into a cell in MRSA.

As set forth above, according to exemplary embodiments of the invention, the phlorotanninn compound according to the present invention exhibits an antibiotic activity against antibiotic-resistant bacteria. In addition, the compound according to the present invention can exhibit a synergetic effect by using in combination with β-lactam-based antibiotic composition by recovering an antibiotic activity of β-lactam-based antibiotic, such as ampicillin, penicillin, and oxacillin against MRSA. Therefore, the antibiotic composition according to the present invention may be used for various purposes and uses requiring an antibiotic activity. Specifically, it can be used for compositions for preventing or treating a disease caused by a pathogenic microbial infection, compositions for animal feeds, additives for animal feeds, foods, food additives, drinks, cosmetic compositions, cosmetic additives, or a cleansing agent. Especially, since Eisenia bicyclis is a natural material and edible, the composition according to the present invention including the compound derived from the same as an effective component has an advantage in that compounds derived from the same is stable even if it is used for a long time.

While the present invention has been shown and described in connection with the exemplary embodiments, it will be apparent to those skilled in the art that modifications and variations can be made without departing from the spirit and scope of the invention as defined by the appended claims.

What is claimed is:

1. An antibiotic composition comprising at least one of phlorotannin compound selected from the group consisting of 7-phloroeckol, fucofuroeckol-A, and dioxinodehydroeckol compound as an effective component.

2. The antibiotic composition according to claim 1, wherein the phlorotannin compound is obtained from extract, fractions, microbial fermentation materials, or purified materials of Eisenia bicyclis.

3. The antibiotic composition according to claim 1, wherein the phlorotannin compound is obtained from a fraction selected from the group consisting of n-hexane fractions, dichloromethane fractions, ethyl acetate fractions, and n-butanol fractions of methanol crude extract of Eisenia bicyclis through an isolation and purification.

4. The antibiotic composition according to claim 1, wherein the phlorotannin compound is obtained from ethyl acetate fractions of methanol crude extract of Eisenia bicyclis through an isolation and purification.

5. The antibiotic composition according to claim 1, wherein the phlorotannin compound has an antibiotic activity against methicillin-resistant Staphylococcus aureus (MRSA).

6. The antibiotic composition according to claim 5, wherein the phlorotannin compound inhibits a synthesis of PBP2a protein in the methicillin-resistant Staphylococcus aureus.

7. The antibiotic composition according to claim 6, further comprising β-lactam-based antibiotics.

8. The antibiotic composition according to claim 1, wherein the antibiotic composition is effective components of pharmaceutically, cosmetically, and foodially composition.

9. The antibiotic composition according to claim 2, wherein the antibiotic composition is effective components of pharmaceutically, cosmetically, and foodially composition.

10. The antibiotic composition according to claim 3, wherein the antibiotic composition is effective components of pharmaceutically, cosmetically, and foodially composition.

11. The antibiotic composition according to claim 4, wherein the antibiotic composition is effective components of pharmaceutically, cosmetically, and foodially composition.

12. The antibiotic composition according to claim 5, wherein the antibiotic composition is effective components of pharmaceutically, cosmetically, and foodially composition.

13. The antibiotic composition according to claim 6, wherein the antibiotic composition is effective components of pharmaceutically, cosmetically, and foodially composition.

14. The antibiotic composition according to claim 7, wherein the antibiotic composition is effective components of pharmaceutically, cosmetically, and foodially composition.
